(19) United States
(12) Patent Application Publication
(10) Pub. No.: US 2010/0168533 A1
(43) Pub. Date: Jul. 1, 2010

(54) SYSTEM AND A METHOD FOR GENERATING A QUANTITATIVE MEASURE REFLECTING THE SEVERITY OF A MEDICAL CONDITION

(75) Inventors: Kristinn Johnsen, Reykjavik (IS); Steinn Gudmundsson, Reykjavik (IS)

Correspondence Address: FOLEY AND LARDNER LLP;SUITE 500, 3000 K STREET NW, WASHINGTON, DC, 20007, US

(73) Assignee: Mentis Cura EHF

(21) Appl. No.: 12/663,030

(22) PCT Filed: Jun. 9, 2008
(86) PCT No.: PCT/EP08/57160

§ 371 Date: Feb. 24, 2010

Related U.S. Application Data
(60) Provisional application No. 60/942,548, filed on Jun. 7, 2007.

(30) Foreign Application Priority Data
Jun. 7, 2007 (EP) 07011207.3

(51) Int. Cl. A61B 5/00

(52) U.S. Cl. 600/301

(57) ABSTRACT

This invention relates to a method and a system for generating a quantitative measure reflecting the severity of a medical condition. A receiver unit receives biosignal data collected from a population of patients having varying degrees of the medical condition. A processor uses the biosignal data for determining reference feature values for each respective patient within the population, where the determining being made in accordance to a pre-defined set of reference features. The processor then assigns each respective patient within the population of patients with a reference feature vector having as vector elements the reference feature values associated with the patient. The processor also uses the reference feature vectors of the patients as input in determining combinations of features describing the variance in the data, where the size of the combinations is an indicator for the severity of the medical condition. This invention further relates to a method and a system for using the quantitative measure for determining a success indicator for a probe compound by implementing the quantitative measure, where a receiver unit receives biosignal data collected from a test subject posterior to administering the probe compound to the test subject, and a processor determines an analogous feature vector as determined for the population of patients. Finally, the processor determines the scalar product between the feature vector determined for the test subject and the combinations of features describing the variance in the data. This scalar product is the success indicator telling how successful the probe compound is.

### FIELD OF THE INVENTION

The present invention relates to a system for generating a quantitative measure reflecting the severity of a medical condition. The present invention further relates to a success monitoring system and a method for determining a success indicator for at least one probe compound by implementing the quantitative measure.

### BACKGROUND OF THE INVENTION

Dementia of the Alzheimer's (AD) type is the most common form of dementia in the elderly. The diagnosis of Alzheimer's Disease is mostly based on standardized clinical criteria (Small et al JAMA 1997). The cornerstone of diagnosis is a detailed history of symptoms from the patient and from a relative with the help of neuroradiological methods (CT, MRI, SPECT, PET) which are quantitative and neuropsychology which is subjective. The accuracy of the clinical diagnosis of AD in mildly or moderately impaired patients is fairly good.

WO 2006/094797 discloses a method and a system for generating a discriminatory signal for a neurological condition, where at least one probe compound that has a neurophysiological effect is provided. This reference may be divided into two parts. One part where a reference distribution is defined and another part where the reference distribution is used for generating a discriminatory signal, i.e. to find out whether a subject suffers from a particular disease.

In part one, data are collected from reference candidates within a given group suffering from a particular disease (e.g. a group of Alzheimer's, this could just as well be a group of healthy subjects) and used for defining a reference tool. This is done by applying the following steps; defining a feature property domain V that contains as domain elements various combinations of the features. As an example if the number of features is three, f1, f2 and f3, the feature property domain V could e.g. be defined by: V={(f1,f2);(f1,f3);(f2,f3)}, where f1,f2 and f3 could be the absolute delta power, absolute theta power and absolute alpha power. For each respective subject within a given group (e.g. group A), a posterior probability vector P={p(f1,f2);p(f1,f3);p(f2,f3)} is calculated in accordance to the domain element. The vector elements of the posterior probability vector indicate the likelihood on whether a particular reference subject belongs to a given group, e.g. group A, with respect to feature property domain V. A filtering process is now performed where those vectors or vector elements that are above or below a pre-defined threshold value are removed. The threshold could e.g. be selected as “0.7”. Thus, if for a given subject within group A, the posterior probability vector gives P={0.9;0.8;0.95}, this indicate that this particular subject is a promising candidate to be used in the reference distribution, whereas a reference subject having P={0.9;0.1;0.5} would not be considered as a potential candidate (or at least not the last two element of P). Such a filtering process is performed for all the candidates within a given reference group (e.g. a group of subjects suffering from Alzheimer disease). After performing such a filtering process for all subjects within e.g. group A, the subjects that have similar characteristics with respect to the domain elements (f1,f2);(f1,f3);(f2,f3) are selected out. The reference tool is thus a reference distribution where the x-axis is domain elements V (i.e. (f1,f2);(f1,f3);(f2,f3)) and on the y-axis the probabilities that the subjects belong to (f1,f2);(f1,f3);(f2,f3), respectively. Thus, a “domain” is formed consisting of a distribution for these three x-values.

In the second part, similar biosignal data is measure for a test subject/patient as for the test subjects. Similar calculations are performed, i.e. a posterior probability vector P={p(f1,f2);p(f1,f3);p(f2,f3)} is calculated. Finally, the values of P are compared to the distribution for the reference subjects, i.e. it is checked whether the values of P lie within the distribution discussed here above. If e.g. all the elements of P lie within this distribution, it is highly likely that this test subject belongs to group A, e.g. has Alzheimer's. If only part of the elements of P lie within this distribution that could indicate that this subject should be examined further.

The result of WO 2006/094797 is that a subject suffering from a neurological condition can be diagnosed earlier than other prior art methods. Thus, the likelihoods of curing the neurological condition or preventing that the neurological condition becomes more severe.

However, WO 2006/094797 does not indicate in any way whether a particular therapy is successful or not.

In order to develop therapies and to be able to monitor the success of such therapies, a measure of the severity of the disease is needed. For instance, if a drug development company has several drug candidates and it needs to choose among the candidates, comparison of the efficacy of the candidates is necessary.

The severity of the disease is determined according to the severity of cognitive impairment of the subject. No recognized quantitative measure exists for this purpose. One way to estimate the severity of AD is by way of the mini mental state examination (MMSE). The test is sensitive to faculties such as short term memory, the ability to follow simple instructions, performance in solving simple problems, awareness of time and place etc. The test results in a numerical score in the range 0-30.

The main problem with such evaluation of the severity of the disease is that it is symptomatic and subjective. It is not linked directly to the physiological pathology of the disease. This means that the evaluation depends on the social and environmental background of the subjects. For instance, Alzheimer's patients with long schooling tend to have higher MMSE scores than patients which have only received elementary education. The outcome of the MMSE is also dependent on the day form of the subject. Yet another problem with such test is that the patient may learn the procedure and answers of the test if the testing is repeated as is the case when therapies are monitored or developed. As a result of these deficiencies drug development and development of other treatments calls for extremely extensive clinical trials in order to obtain the sufficient statistical significance necessary.

### BRIEF DESCRIPTION OF THE INVENTION

The object of the present invention is to overcome the above mentioned drawbacks by providing a system and a method providing a quantitative measure that is sensitive to the physiology of the pathology of a medical condition.

According to one aspect the present invention relates to a system for generating a quantitative measure reflecting the severity of a medical condition, comprising:
a receiver unit for receiving biosignal data collected from a population of M patients, the population being selected such that the patients have varying degrees of a medical condition,
a processor adapted to:
   use the biosignal data as input for determining reference feature values for each respective patient within said population, the determining being made in accordance to a pre-defined set of reference features [f₁, . . . , f_{N}] and results in reference feature vectors F_{1 . . . M}=[value(f₁), value(f₂), . . . , value(f_{N})], the reference feature vectors of the patients subsequently being organized into a M×N matrix A, and
   transform the matrix A into uncorrelated linear combinations of the features x₁·fₛ+x₂·fₚ . . . xₙ·fₜ where indexes x₁ . . . , xₙ describe the variance in the data and wherein the size of the indexes x₁ . . . , xₙ indicate the severity of the medical condition.

It follows that a quantitative measure is provided reflecting the severity of the medical condition, instead of a qualitative measure. Thus, the reliability of using this measure for reflecting the severity of a medical condition becomes very high. As an example, for four different features (e.g. the biosignal data is EEG data and feature 1 is the absolute theta power; feature 2 is the total entropy; feature 3 is the relative gamma power; feature 4 is the peak frequency) and e.g. a population of 40 patients, the matrix A would consist of four columns and 40 lines (or vice verse). The linear combinations of the features could be: 0.7_{absolute gamma power}; 0.15_{total entropy}; 0.10_{absolute theta power}; 0.05_{peak frequency}. This combination shows the line-up of variance of the said pre-set of features. This states that the feature that is mostly influenced by the pathology of a particular disease (e.g. Alzheimer) is the absolute gamma power, the one that is secondly most influenced is the total entropy etc.

In one embodiment, the medical condition is a neurological condition.

In one embodiment, the neurological condition is an Alzheimer's type (AD group).

In one embodiment, the neurological condition is selected from:
Alzheimer's disease,
multiple sclerosis,
mental conditions including depressive disorders,
bipolar disorder and schizophrenic disorders,
Parkinson's disease,
epilepsy, migraine,
Vascular Dementia (VaD),
Fronto-temporal dementia,
Lewy bodies dementia,
Creutzfeld-Jacob disease,
vCJD (“mad cow” disease), and
AD/HD (Attention Deficit/Hyperactive Disorder).

In one embodiment, the receiver is adapted to be coupled to an electroencephalographic (EEG) measuring device and wherein the received data are electroencephalographic (EEG) data.

In one embodiment, the receiver is adapted to be coupled to at least one measuring device selected from:
magnetic resonance imaging (MRI),
functional magnetic resonance imaging (FMRI),
magneto-encephalographic (MEG) measurements,
positron emission tomography (PET),
CAT scanning (Computed Axial Tomography),
single photon emission computerized tomography (SPECT),
a combination of one or more of said measuring devices
   and wherein the biosignal data are the measuring data from one or more of said devices.

In one embodiment, said pre-defined set of reference features is selected from:
the absolute delta power,
the absolute theta power,
the absolute alpha power,
the absolute beta power,
the absolute gamma power,
the relative delta power,
the relative theta power,
the relative alpha power,
the relative beta power,
the relative gamma power,
the total power,
the peak frequency,
the median frequency,
the spectral entropy,
the DFA scaling exponent (alpha band oscillations),
the DFA scaling exponent (beta band oscillations) and
the total entropy.

In one embodiment, determining said combinations of features describing said variance in data comprises means for employing principal component analyses (PCA).

According to another aspect, the present invention relates to a method of generating a quantitative measure reflecting the severity of a medical condition, comprising:
receiving bio signal data collected from a population of patients having varying degrees of the medical condition,
using the bio signal data as input for determining reference feature values for each respective patient within said population, the determining being made in accordance to a pre-defined set of reference features [f₁, . . . , f_{N}] and results in reference feature vectors F_{1 . . . M}=[value(f₁), value(f₂), . . . , value(f_{N})], the reference feature vectors of the patients subsequently being organized into a M×N matrix A,
transforming the matrix A into uncorrelated linear combinations of the features x₁·fₛ+x₂·fₚ . . . xₙ·fₜ where indexes x₁ . . . , xₙ describe the variance in the data and wherein the size of the indexes x₁ . . . , xₙ indicate the severity of the medical condition.

In one embodiment, the method further comprises performing a correlation related measure on the combinations of features describing the variance in the data by comparing said combinations of features describing the variance in the data with an existing measure. In one embodiment, the existing measure is mini mental state examination (MMSE) measure.

Thus, it is possible to optimize the performance of the quantitative measure in relation to the existing one.

In one embodiment, the step of performing a correlation related measure comprises:
repetitively, removing parts from said combinations of features describing the variance in the data or changing the combination of the features describing the variance in the data, and subsequently
determining the correlation between the out-coming combinations of features describing the variance in the data and the existing measure,
   wherein those removed parts that do not contribute to the correlation or lower the correlation are excluded from the combinations of features describing the variance in the data.

In one embodiment, the step of determining combinations of features describing the variance in the data is done using principal component analyses (PCA) and wherein the combinations of features describing the variance in the data is the resulting PCA vector.

According to still another aspect, the present invention relates to a computer program product for instructing a processing unit to execute the method of generating a quantitative measure reflecting the severity of a medical condition when the product is run on a computer.

According to yet another aspect, the present invention relates to a success monitoring system (300) for determining a success indicator for at least one probe compound by implementing the quantitative measure determined by said system, comprising:
a receiver unit for receiving biosignal data collected from a test subject posterior to administering said at least one probe compound to the test subject,
a processor adapted to:
   determine an analogous feature vector F_{1 . . . M}=[test_subj(f₁), test_subj(f₂), . . . , test_subj(f_{N})] for the test subject as determined for said population of M patients, and
   determine the scalar product between the feature vector F_{1 . . . M}=[test_subj(f₁), test_subj(f₂), . . . , test_subj(f_{N})] determined for the test subject and said combinations of features x₁·fₛ+x₂·fₚ . . . xₙ·fₜ describing the variance in the data, the scalar product being the success indicator.

According to yet another aspect, the present invention relates to a method of using said quantitative measure reflecting the severity of a medical condition in determining a success indicator for at least one probe, comprising:
receiving bio signal data collected from a test subject posterior to administering said at least one probe compound to the test subject,
determining an analogous feature vector F_{1 . . . M}=[test_subj(f₁), test_subj(f₂), . . . , test_subj(f_{N})] for the test subject as determined for said population of M patients, and
determining the scalar product between the feature vector F_{1 . . . M}=[test_subj(f₁), test_subj(f₂), . . . , test_subj(f_{N})] determined for the test subject and said combinations of features x₁·fₛ+x₂·fₚ . . . xₙ·fₜ describing the variance in the data, the scalar product being the success indicator.

In order to develop therapies and to be able to monitor the success of such therapies, a measure of the severity of the disease is needed. This system and method accordingly provide a good measure of the severity of a particular disease. In situations where a drug development company has several drug candidates and it needs to choose among the candidates, comparison of the efficacy of the candidates is necessary.

According to yet another aspect, the present invention relates to a computer program product for instructing a processing unit to execute the method of using said quantitative measure reflecting the severity of a medical condition in determining a success indicator for at least one probe when the product is run on a computer.

The aspects of the present invention may each be combined with any of the other aspects. These and other aspects of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows a system according to the present invention system for generating a quantitative measure reflecting the severity of a medical condition,

FIG. 2 shows a flow chart of a method according to the present invention to generate a quantitative measure reflecting the severity of a medical condition,

FIG. 3 shows a success monitoring system according to the present invention for determining a success indicator for at least one probe compound by implementing the quantitative measure discussed under FIGS. 1 and 2,

FIG. 4 shows a flow chart of a method according to the present invention using said quantitative measure discussed in FIGS. 1 and 2 in determining a success indicator for at least one probe compound, and

FIG. 5 is plot showing eigenvectors (pc1) of patients plotted against MMSE score.

### DESCRIPTION OF EMBODIMENTS

FIG. 1 shows a system 100 according to the present invention for generating a quantitative measure reflecting the severity of a medical condition. The system comprises a receiver unit (R) 102 for receiving biosignal data collected from a population of patients 101 having varying degrees of the medical condition. The importance of having varying degrees of the medical condition is to obtain a certain level of a distribution of degrees levels.

In one embodiment, the biosignal data are electroencephalographic (EEG) data. The data could also include biosignal data resulting from one or more of the following measuring devices: magnetic resonance imaging (MRI), functional magnetic resonance imaging (FMRI), magneto-encephalographic (MEG) measurements, positron emission tomography (PET), CAT scanning (Computed Axial Tomography) and single photon emission computerized tomography (SPECT).

In one embodiment, the medical condition is a neurological condition, as an example Alzheimer's type (AD group), multiple sclerosis, mental conditions including depressive disorders, bipolar disorder and schizophrenic disorders, Parkinson's disease, epilepsy, migraine, Vascular Dementia (VaD), Fronto-temporal dementia, Lewy bodies dementia, Creutzfeld-Jacob disease, vCJD (“mad cow” disease) and AD/HD.

The system further comprises a processor (P) 103 adapted to use the biosignal data for determining reference feature values for each respective patient within said population, the determining being made in accordance to a pre-defined set of reference features.

In one embodiment, the pre-defined set of reference features is selected from the absolute delta power, the absolute theta power, the absolute alpha power, the absolute beta power, the absolute gamma power, the relative delta power, the relative theta power, the relative alpha power, the relative beta power, the relative gamma power, the total power, the peak frequency, the median frequency, the spectral entropy, the DFA scaling exponent (alpha band oscillations), the DFA scaling exponent (beta band oscillations) and the total entropy. Accordingly, the pre-defined set of reference features could e.g. be [the absolute theta power; the absolute gamma power; the relative gamma power; the peak frequency]. The determining reference feature values for each respective patient could accordingly be [value 1 (the absolute theta power); value 2 (the absolute gamma power); value 3 (the relative gamma power); value 4 (the peak frequency)].

The processor (P) 103 is further adapted to assign, for each respective patient within said population of patients 101, with a reference feature vector having as vector elements the reference feature values associated with the patient, namely (referring to the example above): vector=[value 1 for the absolute theta power; value 2 for the total entropy; value 3 for the relative gamma power; value 4 for the peak frequency]. The result thereof is a matrix A, where each line indicates the assigned vector for each respective patient. If the number of patient is 40, the number of lines in the matrix is 40.

$$A = [ \begin{matrix}value 1 ( pat .1 ) ; value 2 ( pat .1 ) ; value 3 ( pat .1 ) ; value 4 ( pat .1 ) \\ value 1 ( pat .2 ) ; value 2 ( pat .2 ) ; value 3 ( pat .2 ) ; value 4 ( pat .2 ) \\ value 1 ( pat .3 ) ; value 2 ( pat .3 ) ; value 3 ( pat .3 ) ; value 4 ( pat .3 ) \\ etc .\end{matrix} ]$$

Subsequently, the processor (P) 103 uses the reference feature vectors of the patients as input in determining combinations of features describing the variance in the data 104, where the size of the combinations is an indicator for the severity of the medical condition. As an example, the processor could implement principal component analysis (PCA) for determining the eigenvectors and the values of the covariance matrix of the matrix A, where the result would be a set of uncorrelated linear combinations of the features with eigenvalues relating to the variation in the data. Thus, the result is a linear transformation that chooses a new coordinate system for the data set such that the greatest variance by any projection of the data set comes to lie on the first axis (then called the first principal component), the second greatest variance on the second axis, and so on.

Referring to the example here above, where the matrix A has four different features (the number of columns in the matrix), the resulting combination of features could be: C=[0.7_{absolute gamma power}; 0.15_{total entropy}; 0.10_{absolute theta power}; 0.05_{peak frequency}]. This combination, or a quantitative measure vector C 104 shows the line-up of variance of said pre-set of features (referring to the example above). This states that the feature that is mostly influenced by the pathology of a particular disease (e.g. Alzheimer) is the absolute gamma power, the one that is secondly most influenced is the total entropy etc.

In one embodiment, the receiver unit (R) 102 is coupled to at least one measuring device 106. These could e.g. be a electroencephalograph (EEG), magnetic resonance imaging (MRI), a functional magnetic resonance imaging (FMRI), a magneto-encephalographic (MEG) measurements, a positron emission tomography (PET), a CAT scanning (Computed Axial Tomography), a single photon emission computerized tomography (SPECT), a combination of one or more of said measuring devices and the like. The receiver unit (R) 102 could also be adapted to be coupled to an external memory 105 over a communication channel.

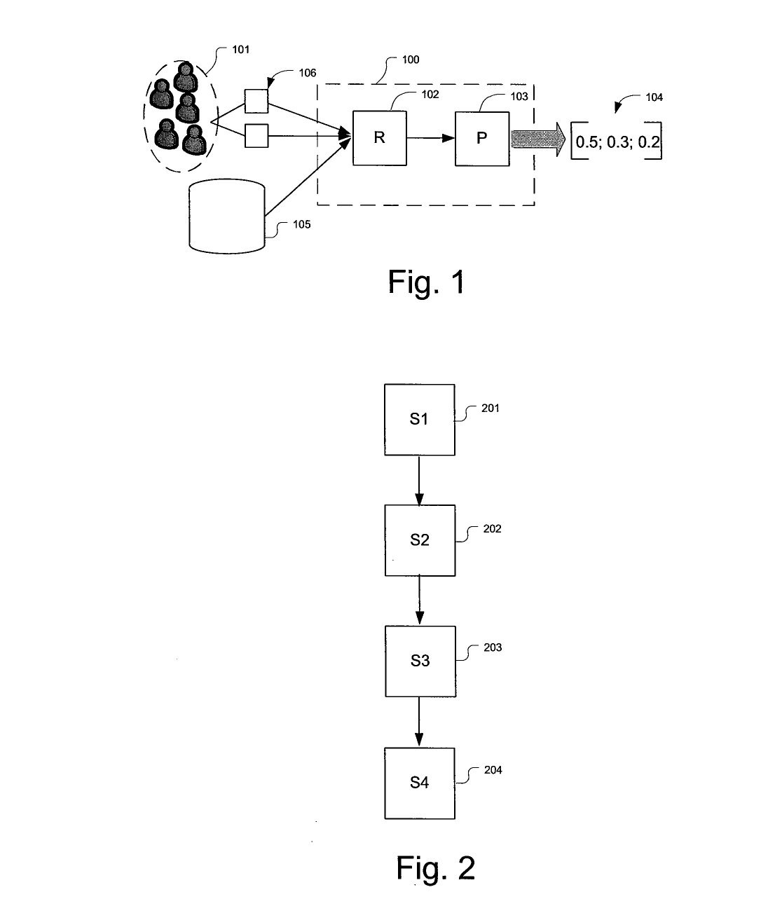

FIG. 2 shows a flow chart of a method according to the present invention to generate a quantitative measure reflecting the severity of a medical condition.

In one embodiment, the method includes receiving biosignal data (S1) 201 collected from a population of patients having varying degrees of the medical condition, using the biosignal data (S2) 202 for determining reference feature values for each respective patient within said population, the determining being made in accordance to a pre-defined set of reference features. The method further includes assigning each respective patient within said population of patients with a reference feature vector (S3) 203 having as vector elements the reference feature values associated with the patient, and using the reference feature vectors of the patients as input in determining combinations of features describing the variance in the data (S4) 204, the size of the combinations being an indicator for the severity of the medical condition.

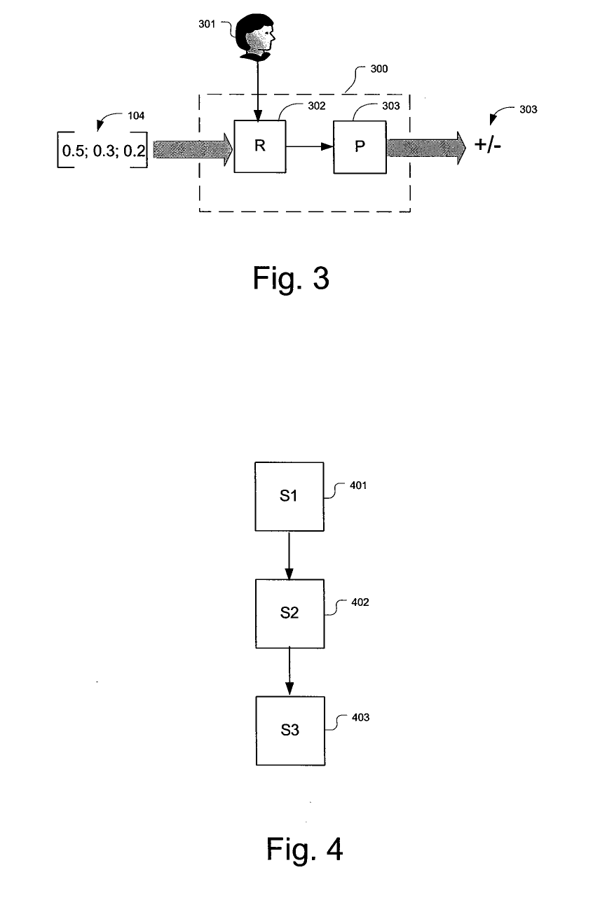

FIG. 3 shows a success monitoring system 300 according to the present invention for determining a success indicator 303 for at least one probe compound by implementing the quantitative measure discussed under FIGS. 1 and 2. The success monitoring system comprises a receiver unit (R) 302 for receiving bio signal data collected from a test subject 301 posterior to administering said at least one probe compound to the test subject 301 and a processor (P) 303 for determining an analogous feature vector as determined for said population of patients. The processor (P) 303 is further implemented for determining the scalar product between the feature vectors determined for the test subject and said combinations of features describing the variance in the data, the scalar product being an indicator of the success indicator.

Referring to the example given above, vector=[value 1 for the absolute theta power; value 2 for the total entropy; value 3 for the relative gamma power; value 4 for the peak frequency], which is simply a four dimension vector. This vector is multiplied with said C=[0.7_{absolute gamma power}; 0.15_{total entropy}; 0.10_{absolute theta power}; 0.05_{peak frequency}] vector which gives a certain value, here referred as +/−303. It is precisely this value 303 which provides a very good indication whether the probe compound, e.g. any kind of a new medicine, is successful or not in treating or curing a particular disease.

FIG. 4 shows a flow chart of a method according to the present invention using said quantitative measure discussed in FIGS. 1 and 2 in determining a success indicator for at least one probe compound.

In one embodiment, the method includes receiving biosignal data collected from a test subject (S1) 401 posterior to administering said at least one probe compound to the test subject, determining an analogous feature vector as determined for said population of patients (S2) 402, and determining the scalar product between the feature vector determined for the test subject and said combinations of features describing the variance in the data (S3) 403, the scalar product being an indicator of the success indicator.

Establishment of the Quantitative Measure:

In order to establish that a quantitative physiological measure reflects the severity of a certain disease, it is necessary to demonstrate that the measure correlates with existing measures that are sensitive to the severity, even if no gold standard exists and that measure is subjective or indirect. One way to look for quantitative measures is to establish a database of features obtained from physiological measurements collected from a population of patients subject to varying degrees of the disease. Thus the subjects in the database do not represent a uniform population and one would expect a degree of variation in the data related to the severity of the disease if the physiological data or part of it is sensitive to the relevant pathology. In case that the data reflects the state of the patient well, one would expect that the majority of the variation of the data in the database is due to the varying degree of the disease. If that is the case, factor analysis such as principal component analysis (PCA), eigenvectors and values of the correlation matrix of the features, will reveal uncorrelated linear combination of the features that describe the variation in the data. Then the principal component with the largest eigenvalue describes the largest variation in the data and will be correlated to the severity of the disease. If this is the case it can then be verified by estimating the correlation between the existing measure and the principal component found from the database. Note that the correlation is not necessarily high. If the existing measure is subjective and subject to influence from external condition that are not related to the disease, such as is the case with MMSE (mini mental state examination) scores for the state of Alzheimer's patients, one simply has to establish a significant finite correlation in order to identify the quantitative measure. After that, independent tests or clinical trials must be conducted in order to determine the quality of the new quantitative measure. Using the strategy described above, one can optimize the performance of the new measure by repeatedly excluding parts of the data that does not contribute to, or even decreases, the correlation between the existing measure and the new quantitative measure. By following such a procedure systematically, for instance using a genetic algorithmic approach or simply by testing all combinations, one can optimize the performance of the new measure in relation to the existing one.

### Example

### Alzheimer's Disease

Electroencephalography (EEG) records the electrical activity of the brain. The activity contains information about the state of the brain. EEG is physiological, so when the pathology of a particular disease, such as Alzheimer's disease, affects the EEG it becomes a candidate as a quantitative measure that is sensitive to the severity of the disease.

A clinical trial was conducted in order to establish a database of features. A group of 60 Alzheimer's patients with varying degrees of the disease was recruited and the EEG was recorded on each of the patients. Three minutes of recording was collected from each subject while the patient kept his eyes closed and was at rest. The electroencephalographic signals were recorded using computerized measuring equipment. The recordings were performed using the conventional International 10-20 system of electrode placement. The collected data is stored in raw format on a storage device for later analysis. During the recordings the signals are displayed simultaneously on a computer screen. This allows the operator to monitor if electrodes come loose and to enter marks that indicate specific events. Such events may indicate initiation of specific parts of the recording protocol or occurrences that may lead to artifacts being present in the recordings. Such occurrences include that the subject blinks his eyes, swallows, moves or in general breaches protocol. Influences from such events were excluded during extraction of the features. The features were extracted using 40 seconds of artifact free recordings. Features extracted were derived from results reported in the scientific literature (Adler G. et al. 2003, Babiloni C. et al. 2004, Bennys K. et al. 2001, Brunovsky M. et al. 2003, Cichocki et al. 2004, Cho S. Y. 2003, Claus J. J. et al. 1999, Hara J. et al. 1999, Holschneider D. P. et al. 2000, Hongzhi Q. I. et al. 2004, Huang C. et al. 2000, Hyung-Rae K. et al. 1999, Jelles B. et al. 1999, Jeong J. et al. 1998, 2001, 2004, Jonkman E. J. 1997, Kikuchi M. et al. 2002, Koenig T. et al. 2004, Locatelli T. et al. 1998, Londos E. et al. 2003, Montplaisir J. et al. 1998, Moretti et al. 2004, Musha T. et al. 2002, Pijnenburg Y. A. L. et al. 2004, Pucci E. et al. 1998, 1999, Rodriquez G. et al. 1999, Signorino M. et al. 1995, Stam C. J. et al. 2003, 2004, Stevens A. et al. 1998, 2001, Strik W. K. et al. 1997, Vesna J. et al. 2000, Wada Y. et al. 1998, Benvenuto J. et al. 2002, Jimenez-Escrig A. et al. 2001, Sumi N. et al. 2000), hereby incorporated as whole by reference. The features used in the example were numbered as follows. 16 base features were selected and extracted from each channel.
1.—Absolute delta power
2.—Absolute theta power
3.—Absolute alpha power
4.—Absolute beta power
5.—Absolute gamma power
6.—Relative delta power
7.—Relative theta power
8.—Relative alpha power
9.—Relative beta power
10.—Relative gamma power
11.—Total power
12.—Peak frequency
13.—Median frequency
14.—Spectral entropy
15.—DFA scaling exponent (alpha band oscillations)
16.—DFA scaling exponent (beta band oscillations)

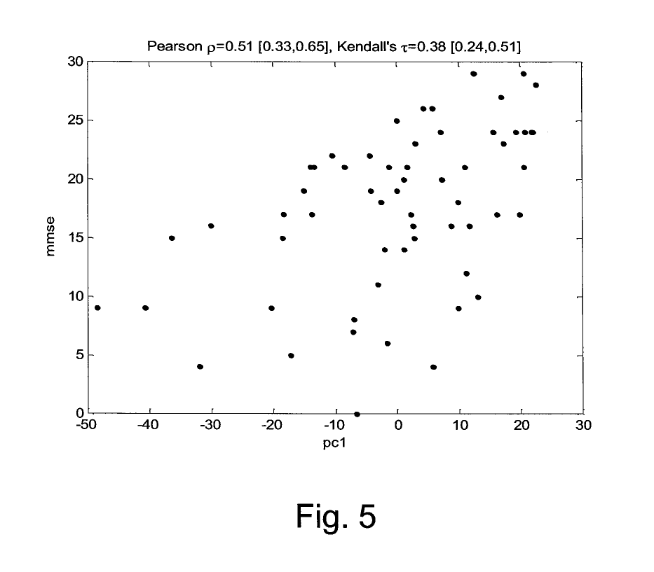

The data collected into the database was organized into a matrix X where each row contained all the features extracted from the recordings of a particular patient. Thus the dimension of the matrix was the number of subjects times the number of features extracted. Principal component analysis was then performed on X. After that the principal component with the largest eigenvalue (pc1) of each subject was plotted against the MMSE score of the same subject. Evident from FIG. 5 is that a trend exists between pc1 and MMSE. Pearson's linear correlation coefficient, ρ, and Kendall's τ were evaluated in order to establish the correlation between pc1 and MMSE. It was found that ρ=0.51[0.33,0.65] and τ=0.38[0.24,0.51] where the 2 standard deviations were estimated using the bootstrap resampling method. It was established that the correlation between pc1 and MMSE is significant and thereby that pc1 is a quantitative measure that correlates with the severity of the disease.

We have thus found a quantitative measure, based on EEG recordings and a database of patient data, which is able to track the progress of the Alzheimer's disease.

Certain specific details of the disclosed embodiment are set forth for purposes of explanation rather than limitation, so as to provide a clear and thorough understanding of the present invention. However, it should be understood by those skilled in this art, that the present invention might be practiced in other embodiments that do not conform exactly to the details set forth herein, without departing significantly from the spirit and scope of this disclosure. Further, in this context, and for the purposes of brevity and clarity, detailed descriptions of well-known apparatuses, circuits and methodologies have been omitted so as to avoid unnecessary detail and possible confusion.

Reference signs are included in the claims, however the inclusion of the reference signs is only for clarity reasons and should not be construed as limiting the scope of the claims.

### REFERENCE LIST

Adler G. et al. EEG coherence in Alzheimer's dementia. Journal of neural transmission, Vol 110, no. 9, 2003.
Babiloni, C. et al. Mapping distributed sources of cortical rhythms in mild Alzheimer's disease. A multicentric EEG study. Neuroimage 22 57-67, 2004.
Babiloni, C. et al. Abnormal fronto-parietal coupling of brain rhythms in mild Alzheimer's disease: a multicentric EEG study. European Journal of Neuroscience, Vol. 19, pp. 2583-2590 2004.
Bennys, K. et al. Diagnostic value of quantitative EEG in Alzheimer's disease. Neurophysiol Clin, 31, 153-60, 2001.
Benvenuto J. et al. Identification of diagnostic evoked response potential segments in Alzheimer's disease. Experimental Neurology 176, 269-276, 2002.
Brunovsky M., Matousek M. et al. EEG assessment of the degree of dementia. Neuropsychobiology 48:19-26, 2003.
Cichocki et al. EEG filtering based on blind source separation (BSS) for early detection of Alzheimer's disease. Clinical Neurophysiology, In-press, 2004.
Cho, S. Y. et al. Automatic recognition of Alzheimer's disease using genetic algorithms and neural network. Lecture Notes in Computer Science, Springer-Verlag, Volume 2658, pp. 695-702, 2003.
Claus, J. J. et al. The diagnostic value of electroencephalography in mild senile Alzheimer's disease. Clinical Neurophysiology 110, 825-832, 1999.
Duda R O, Hart P E and Stork D G, Pattern Classification, Wiley-Interscience; 2nd edition, 2000.
Hara J. et al. Cortical atrophy in Alzheimer's disease unmasks electrically silent sulci and lowers EEG dipolarity. IEEE TBME 46, 8, 1999.
Holschneider, D. P. et al. Loss of high-frequency brain electrical response to thiopental administration in Alzheimer's-type dementia. Neuropsychopharmacology 16, 4, 1997.
Holschneider, D. P. et al. Attenuation of brain high frequency electrocortical response after thiopental in early stages of Alzheimer's dementia. Psychopharmacology, 149, 6-11, 2000.
Hongzhi, Q. I. et al. Mutual information entropy research on dementia EEG signals. The Fourth International Conference on Computer and Information Technology (CIT '04), Sep. 14-16, 2004, Wuhan, China.
Huang, C. et al. Discrimination of Alzheimer's disease and mild cognitive impairment by equivalent EEG sources: a cross-sectional and longitudinal study. Clinical Neurophysiology 111, 1961-1967, 2000.
Hyung-Rae K. et al. Analysis of the spatiotemporal EEG pattern in Alzheimer's disease. Journal of the Korean Physical Society, Vol. 34, No. 4, April 1999.
Jelles B. et al. Decrease of non-linear structure in the EEG of Alzheimer patients compared to healthy controls. Clinical Neurophysiology 110, 1159-1167, 1999.
Jeong, J. et al. Non-linear dynamical analysis of the EEG in Alzheimer's disease with optimal embedding dimension. Electroencephalography and clinical Neurophysiology 106, 220-228, 1998.
Jeong, J. et al. Mutual information analysis of the EEG in patients with Alzheimer's disease. Clinical Neurophysiology 112, 827-835, 2001.
Jeong, J. EEG dynamics in patients with Alzheimer's disease. Clinical Neurophysiology, In-press, 2004.
Jimenez-Escrig A. et al. P300 latency reveals cholinergic enhancement produced by rivastigmine in Alzheimer's disease. Alzheimer's Reports, Vol. 4, No. 1, 2001.
Jonkman, E. J. The role of the electroencephalogram in the diagnosis of dementia of the Alzheimer type: an attempt at technology assessment. Neurophysiol Clin, 27, 211-9, 1997.
Kikuchi, M. et al. EEG harmonic responses to photic stimulation in normal aging and Alzheimer's disease: differences in interhemispheric coherence. Clinical Neurophysiology 113, 1045-1051, 2002.
Koenig, T. et al. Decreased EEG synchronization in Alzheimer's disease and mild cognitive impairment. Neurobiology of aging, In press, 2004.
Locatelli, T. et al. EEG coherence in Alzheimer's disease. Electroencephalography and clinical Neurophysiology 106, 229-237, 1998.
Londos, E., Rosen, I. et al. Regional cerebral blood flow and EEG in clinically diagnosed dementia with Lewy bodies and Alzheimer's disease. Arch. Gerontol. Geriatr. 36, 231-245, 2003.
Montplaisir, J. et al. Sleep disturbances and EEG slowing in Alzheimer's disease. Sleep Research Online 1(4): 147-151, 1998
Moretti et al. Individual analysis of EEG frequency and band power in mild Alzheimer's disease. Clinical Neurophysiology 115, 299-308, 2004.
Musha, T. et al. A new EEG method for estimating cortical neuronal impairment that is sensitive to early stage Alzheimer's disease. Clinical Neurophysiology 113, 1052-1058, 2002.
Nikulin V., Brismar T., Long-range temporal correlation in alpha and beta oscillations: effects of arousal level and test-retest reliability. Clinical Neurophysiology 115, 1896-1908, 2004.
Pijnenburg Y. A. L. et al. EEG synchronization likelihood in mild cognitive impairment and Alzheimer's disease during a working memory task. Clinical Neurophysiology, In-press, 2004.
Pucci, E. et al. EEG spectral analysis in Alzheimer's disease and different degenerative dementias. Archives of Gerontology and Geriatrics 26, 283-297, 1998.
Pucci, E. et al. EEG power spectrum differences in early and late onset forms of Alzheimer's disease. Clinical Neurophysiology 110, 621-631, 1999.
Rodriquez, G. et al. EEG spectral profile to stage Alzheimer's disease. Clinical Neurophysiology 110, 1831-1837, 1999.
Shawe-Taylor J and Cristianini N, Kernel Methods for Pattern Analysis, Cambridge University Press, 2004.
Signorino M. et al. EEG spectral analysis in vascular and Alzheimer dementia. Electroencephalography and clinical neurophysiology 94:313-325, 1995.
Sumi, N. et al. Interpeak latency of auditory event-related potentials (P300) in senile depression and dementia of the Alzheimer type. Psychiatry and Clinical Neurosciences, 54, 679-684, 2000.
Stam C. J. et al. EEG synchronization in mild cognitive impairment and Alzheimer's disease. Acta Neurol Scand 108, 90-96, 2003.
Stam C. J. et al. Disturbed fluctuations of resting state EEG synchronization in Alzheimer's disease. Clinical Neurophysiology, In-press, 2004.
Stevens A. et al. Cognitive decline unlike normal aging is associated with alterations of EEG temporo-spatial characteristics. Eur Arch Psychiatry Clin Neurosci 248:259-266, 1998.
Stevens A. et al. Dynamic regulation of EEG power and coherence is lost early and globally in probable DAT. Eur Arch Psychiatry Clin Neurosci 251:199-204, 2001.
Strik, W. K. et al. Decreased EEG microstate duration and anteriorisation of the brain electrical fields in mild and moderate dementia of the Alzheimer type. Psychiatry Research: Neuroimaging Section, 75, 183-191 (1997).
Vesna J. et al. Quantitative electroencephalography in mild cognitive impairment: longitudinal changes and possible prediction of Alzheimer's disease. Neurobiology of aging 21, 533-540, 2000.
Wada Y. et al. Abnormal functional connectivity in Alzheimer's disease: intrahemisphereic EEG coherence during rest and photic stimulation. Eur Arch Psychiatry Clin Neurosci 248:203-208, 1998.

1. A system (100) for generating a quantitative measure reflecting the severity of a medical condition, comprising:
a receiver unit (102) for receiving biosignal data collected from a population of M patients, the population being selected such that the patients have varying degrees of a medical condition,
a processor (103) adapted to:
use the biosignal data as input for determining reference feature values for each respective patient within said population, the determining being made in accordance to a pre-defined set of reference features [f₁, . . . , f_{N}] and results in reference feature vectors F_{1 . . . M}=[value(f₁), value(f₂), . . . , value(f_{N})], the reference feature vectors of the patients subsequently being organized into a M×N matrix A, and
transform the matrix A into uncorrelated linear combinations of the features x₁·fₛ+x₂·fₚ . . . xₙ·fₜ where indexes x₁ . . . , xₙ describe the variance in the data and wherein the size of the indexes x₁ . . . , xₙ indicate the severity of the medical condition.

2. A system according to claim 1, wherein the medical condition is a neurological condition.

3. A system according to claim 2, wherein the neurological condition is an Alzheimer's type (AD group).

4. A system according to claim 2, wherein the neurological condition is selected from:
Alzheimer's disease,
multiple sclerosis,
mental conditions including depressive disorders,
bipolar disorder and schizophrenic disorders,
Parkinson's disease,
epilepsy, migraine,
Vascular Dementia (VaD),
Fronto-temporal dementia,
Lewy bodies dementia,
Creutzfeld-Jacob disease and
vCJD (“mad cow” disease), and
AD/HD (Attention Deficit/Hyperactive Disorder)

5. A system according to claim 1, wherein the receiver is adapted to be coupled to an electroencephalographic (EEG) measuring device (106) and wherein the received data are electroencephalographic (EEG) data.

6. A system according to claim 1, wherein the receiver is adapted to be coupled to at least one measuring device (106) selected from:
magnetic resonance imaging (MRI),
functional magnetic resonance imaging (FMRI),
magneto-encephalographic (MEG) measurements,
positron emission tomography (PET),
CAT scanning (Computed Axial Tomography),
single photon emission computerized tomography (SPECT),
a combination of one or more of said measuring devices
and wherein the biosignal data are the measuring data from one or more of said devices.

7. A system according to claim 1, wherein said pre-defined set of reference features is selected from:
the absolute delta power,
the absolute theta power,
the absolute alpha power,
the absolute beta power,
the absolute gamma power,
the relative delta power,
the relative theta power,
the relative alpha power,
the relative beta power,
the relative gamma power,
the total power,
the peak frequency,
the median frequency,
the spectral entropy,
the DFA scaling exponent (alpha band oscillations),
the DFA scaling exponent (beta band oscillations) and
the total entropy.

8. A system according to claim 1, wherein determining said combinations of features describing said variance in data comprises means for employing principal component analyses (PCA).

9. A method of generating a quantitative measure reflecting the severity of a medical condition, comprising:
receiving biosignal data (201) collected from a population of patients having varying degrees of the medical condition,
using the biosignal data (202) as input for determining reference feature values for each respective patient within said population, the determining being made in accordance to a pre-defined set of reference features [f₁, . . . , f_{N}] and results in reference feature vectors F_{1 . . . M}=[value(f₁), value(f₂), . . . , value(f_{N})], the reference feature vectors of the patients subsequently being organized into a M×N matrix A,
transforming the matrix A into uncorrelated linear combinations of the features x₁·fₛ+x₂·fₚ . . . xₙ·fₜ where indexes x₁ . . . , xₙ describe the variance in the data and wherein the size of the indexes x₁ . . . , xₙ indicate the severity of the medical condition.

10. A method according to claim 9, further comprising performing a correlation related measure on the combinations of features describing the variance in the data by comparing said combinations of features describing the variance in the data with an existing measure.

11. A method according to claim 9, wherein the existing measure is mini mental state examination (MMSE) measure.

12. A method according to claim 10, wherein performing a correlation related measure comprises:
repetitively, removing parts from said combinations of features describing the variance in the data or changing the combination of the features describing the variance in the data, and subsequently
determining the correlation between the out-coming combinations of features describing the variance in the data and the existing measure,
wherein those removed parts that do not contribute to the correlation or lower the correlation are excluded from the combinations of features describing the variance in the data.

13. A method according to claim 9, wherein the step of determining combinations of features describing the variance in the data is done using principal component analyses (PCA) and wherein the combinations of features describing the variance in the data is the resulting PCA vector.

14. A computer program product for instructing a processing unit to execute the method step of claim 9 when the product is run on a computer.

15. A success monitoring system (300) for determining a success indicator for at least one probe compound by implementing the quantitative measure determined by the system according to claim 1, comprising:
a receiver unit (302) for receiving biosignal data collected from a test subject posterior to administering said at least one probe compound to the test subject,
a processor (303) adapted to:
determine an analogous feature vector F_{1 . . . M}=[test_subj(f₁), test_subj(f₂), . . . , test_subj(f_{N})] for the test subject as determined for said population of M patients, and
determine the scalar product between the feature vector F_{1 . . . M}=[test_subj(f₁), test_subj(f₂), . . . , test_subj(f_{N})] determined for the test subject and said combinations of features x₁·fₛ+x₂·fₚ . . . xₙ·fₜ describing the variance in the data, the scalar product being the success indicator.

16. A method of using the quantitative measure reflecting the severity of a medical condition as claimed in claim 9 in determining a success indicator for at least one probe, comprising:
receiving biosignal data (401) collected from a test subject posterior to administering said at least one probe compound to the test subject,
determining an analogous feature vector F_{1 . . . M}=[test_subj(f₁), test_subj(f₂), . . . , test_subj(f_{N})] for the test subject (402) as determined for said population of M patients, and
determining the scalar product between the feature vector F_{1 . . . M}=[test_subj(f₁), test_subj(f₂), . . . , test_subj(f_{N})] determined for the test subject and said combinations of features x₁·fₛ+x₂·fₚ . . . xₙ·fₜ (403) describing the variance in the data, the scalar product being the success indicator.

17. A computer program product for instructing a processing unit to execute the method step of claim 16 when the product is run on a computer.
